(19) United States
(12) Patent Application Publication
(10) Pub. No.: US 2004/0157227 A1
(43) Pub. Date: Aug. 12, 2004

(54) Reproductive cancer diagnosis and therapy

(76) Inventors: Lisa Kerstin Chopin, Brisbane (AU); Penelope Lorrelle Jeffrey, Brisbane (AU); Adrian Charles Herington, Brisbane (AU)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 10/477,506

(22) PCT Filed: May 10, 2002
(86) PCT No.: PCT/AU02/00582

(30) Foreign Application Priority Data
May 10, 2001 (AU) PR 4919

(51) Int. Cl. C12Q 1/68; G01N 33/574

(52) U.S. Cl. 435/6; 435/7.23

(57) ABSTRACT

A method of detecting a cancer cell or tissue of the reproductive system such as prostate cancer, breast cancer, ovarian cancer, cervical cancer and uterine cancer uses detection of relatively increased levels of ghrelin, an exon 3-deleted form of preproghrelin and/or growth hormone secretagogue type 1b receptor expression by cancer cells as compared to normal cells and tissues of the reproductive system. Also provided is an exon 3-deleted form of preproghrelin and antibodies thereto as well interventionist strategies that target ghrelin and/or growth hormone secretagogue receptors in treating cancers of the reproductive system such as prostate cancer and breast cancer, although without limitation thereto.

### FIELD OF THE INVENTION

THIS INVENTION relates to diagnosis and treatment of cancers of the reproductive system such as prostate cancer, breast cancer, ovarian cancer, cervical cancer and uterine cancer. More particularly, this invention relates to detection of ghrelin, an exon 3-deleted form of preproghrelin and growth hormone secretagogue type 1b receptor expression by cancer cells and tissues of the reproductive system, and to interventionist strategies that target ghrelin and/or growth hormone secretagogue receptors in treating cancers of the reproductive system.

### BACKGROUND OF THE INVENTION

Prostatic carcinoma is the most frequent cause of cancer mortality in males in the Western world, with aging identified as the number one risk factor. Insulin-like growth factor-I (IGF-I), the tissue biomediator of growth hormone (GH), has an autocrine/paracrine action in the prostate (Culig et al., 1996, Prostate 28 392) and a positive association has been identified between circulating IGF-I levels and prostate cancer risk (Chan et al., 1998, Science 279 563), although this link has been disputed (Cutting et al., 1999, BJU International 83 996). Other components of the growth hormone (GH) axis may also have tumourigenic potential (Chopin et al., 1999 Clin. Biochemist. Rev. 20 3). Antagonistic analogues of growth hormone releasing hormone (GHRH) inhibit the growth of androgen-independent human prostate cancer cell lines both in vivo and in vitro (Schally & Varga, 1999, Endocrinol. & Metab. 10 383). The expression of GH receptor (GH-R) mRNA has been demonstrated in normal and cancerous human prostate tissue (Ballesteros et al., 2000 J. Clin. Endocrinol. Metab. 85 2865). There have been reports of an increased in vitro proliferation rate in human LNCAP prostate cancer cells in response to GH (Untergasser et al., 1999, Exp. Gerontol. 34 275), and GH receptor (GH-R) antagonists possess antineoplastic properties in numerous tumours (Duan et al., 1999, IGF Res. 9 340; Friend et al., 1999, J. Neurosurg. 91 93).

Synthetic growth hormone secretagogues (GHSs) are potent inducers of GH secretion in humans, as demonstrated in both in vitro and in vivo studies. These peptide and non-peptide molecules were initially developed for clinical use in disease states such as GH deficiency. GHSs exert their effects via activation of the growth hormone secretagogue receptor (GHS-R).

The naturally occurring growth hormone secretagogue receptor (GHS-R) was first cloned in 1996 (Howard et al., 1996, Science 273 974). The human functional GHS-R (type 1a) is a 366 amino acid peptide with seven transmembrane domains, and is a member of the heptahelical superfamily of G protein-coupled receptors (Carnanni et al., 1998, Front. Neuroendocrinol. 19 47). The type 1b splice variant is predicted to encode a 289 amino acid protein lacking two of the transmembrane domains and is regarded as a non-functional receptor (McKee et al., 1997, Mol. Endocrinol. 11 415). A recently identified endogenous ligand for the GHS-R, ghrelin, is a 28 amino acid peptide originally isolated from rat stomach tissue and subsequently from human stomach (Kojima et al., 1999, Nature 402 656). Ghrelin was found to stimulate pituitary GH release in vitro and in vivo with a potency and specificity comparable to Growth Hormone Releasing Hormone (GHRH; Kojima et al., 1999, supra).

GHS-R expression has been associated with certain tumours such as pituitary adenomas and other neuroendocrine tumours (Korbonits et al., 1998, J. Clin. Endocrinol. Metab. 83 3624) and thyroid carcinomas (Cassoni et al., 2000, J. Endocrinol. 165 139). A non-defined ghrelin binding site has been reported in breast cancer tissue which may mediate growth inhibitory effects on breast cancer cell lines in vitro (Cassoni et al., 2001, J. Clin. Endocrinol. Metab. 86 1738).

### OBJECT OF THE INVENTION

The present inventors have unexpectedly discovered expression of ghrelin, GHS-R 1a and GHS-R 1b by cancer cells and tissues of the reproductive system. Furthermore, expression of ghrelin and/or GHS-R 1b distinguishes cancer cells from normal cells, particularly in the case of prostate and breast cells and tissues. The present inventors have also identified a novel, exon 3-deleted form of preproghrelin, the expression of which distinguishes cancer cells and tissues from normal cells and tissues of the reproductive system.

It is therefore an object of the invention to provide a method of detection of cancer cells and tissues of the reproductive system.

It is also an object of the invention to provide therapy for cancers of the reproductive system.

### SUMMARY OF THE INVENTION

In one aspect, the invention provides a method of identifying a cancer cell or tissue of the reproductive system, said method including the step of detecting expression of ghrelin, an exon 3-deleted form of preproghrelin and/or GHS-R 1b by a cell or tissue of the reproductive system, wherein at least the presence of said ghrelin or said GHS-R 1b indicates that said cell or tissue is a cancer cell or tissue.

In one embodiment of this aspect of the invention, expression of ghrelin, an exon 3-deleted preproghrelin and/or GHS-R 1b protein is detected.

In another embodiment of this aspect of the invention, expression of ghrelin, an exon 3-deleted preproghrelin and/or GHS-R 1b nucleic acid is detected.

In one particular embodiment, the expression of GHS-R 1b protein or nucleic acid is detected as an indication that said cell or tissue is a cancer cell or tissue.

In another particular embodiment, the expression of ghrelin protein or nucleic acid is detected as an indication that said cell or tissue is a cancer cell or tissue.

In yet another particular embodiment, an exon 3-deleted form of preproghrelin protein or nucleic acid is detected as an indication that said cell or tissue is a breast cancer or prostate cancer cell or tissue.

Preferably, according to this aspect, expression of ghrelin, exon3-deleted preproghrelin or GHS-R 1b protein or nucleic acid is higher in said cancer cell or tissue than in a corresponding normal cell or tissue of the reproductive system.

In another aspect, the present invention provides an isolated protein that includes the amino acid sequence RPQPTSDRPQALLTSL (SEQ ID NO:1).

In particular embodiments, the isolated protein is an exon 3-deleted form of preproghrelin or proghrelin

Preferably, the exon 3-deleted form of preproghrelin has the amino acid sequence:

| | |
|---|---|
| MPSPGTVCSLLLLGMLWLDLAMAGSSFLSPEHQR | (SEQ ID NO: 2) |
| VQQRKESKKPPAKLQPRALAGWLRPEDGGQAEGA | |
| EDELEVRRPQPTSDRPQALLTSL. | |

The invention also provides an isolated nucleic acid that encodes the exon 3-deleted form of preproghrelin or proghrelin.

Preferably, the isolated nucleic acid has the nucleotide sequence shown in FIG. 2 (SEQ ID NO:3).

In yet another aspect, the invention provides a method of treating cancer of the reproductive system, said method including the step of administering to an individual an agent that suppresses or inhibits ghrelin activity.

By “ghrelin activity” is meant the biological activity of any component of the ghrelin system. That is, ghrelin itself, GHS-R 1a or GHS-R 1b and homologous or structurally and functionally related ligands or receptors that are associated with cancers of the reproductive system.

Preferably, suppression or inhibition of ghrelin activity inhibits or reduces cancer cell proliferation, motility and/or invasiveness or promotes cancer cell apoptosis.

The cancer of the reproductive system includes, but is not limited to, prostate cancer, ovarian cancer, breast cancer, cervical cancer, choriocarcinoma and uterine cancer. For the purpose of disease treatment, the present invention also contemplates hyperproliferative disorders of the reproductive system such as benign prostatic hyperplasia.

Preferably, the reproductive cancer is prostate cancer.

Suitably, said individual is a mammal.

Preferably, said individual is a human.

In still yet another aspect, the invention provides an antibody that binds ghrelin, an exon 3-deleted form of preproghrelin, GHS-R 1a or GHS-R 1b.

According to this aspect, the antibody may be useful according to the aforementioned method of detection, or to neutralize ghrelin binding to GHS-R 1a, for example.

In one embodiment, for the purposes of detection, the antibody is a GHS-R 1b-specific antibody.

In another embodiment, the antibody is capable of distinguishing between the aforementioned exon 3-deleted form of preproghrelin and preproghrelin or ghrelin.

Preferably, the antibody is capable of binding the amino acid sequence RPQPTSDRPQALLTSL (SEQ ID NO:1), an antigenic fragment thereof or a larger peptide that includes this sequence.

In a further aspect, the invention provides a method of identifying an antagonist of ghrelin activity, said method including the step of determining whether a candidate molecule inhibits or suppresses ghrelin activity.

In a still further aspect, the invention provides nucleic acids and expression constructs comprising same that may be useful in gene therapy methods of treatment of cancer of the reproductive system.

In one embodiment, said nucleic acid encodes a protein antagonist of ghrelin/GHS-R 1a receptor binding.

In another embodiment, said nucleic acid encodes ghrelin, GHS-R 1a, and/or GHS-R 1b such as may be useful in antisense inhibition of ghrelin and/or GHS-R 1a expression.

In a yet still further aspect, the invention provides a pharmaceutical composition comprising an agent that suppresses ghrelin activity together with a pharmaceutically-acceptable carrier, diluent or excipient.

Throughout this specification, unless the context requires otherwise, the words “comprise”, “comprises” and “comprising” will be understood to imply the inclusion of a stated integer or group of integers but not the exclusion of any other integer or group of integers.

### BRIEF DESCRIPTION OF THE FIGURES AND TABLES

Table 1: Summary of immunohistochemical staining of breast and prostate tissue. Relative levels of expression are indicated as follows: absence of staining=−; mild staining=+; moderate staining=++; strong staining=+++; very strong staining=++++; and strongest staining=+++++.

FIG. 1: Nucleotide sequence of exon 3-deleted preproghrelin mRNA (SEQ ID NO:3) and encoded protein (SEQ ID NO:2). The novel C-terminal peptide sequence RPQPTSDRPQALLTSL (SEQ ID NO:1) is bolded within the SEQ ID NO:2 sequence. The wild type precursor mRNA (SEQ ID NO:4) and encoded preproghrelin protein (SEQ ID NO:5) are also shown.

FIG. 2: Structure of wild type preproghrelin, proghrelin and ghrelin mRNA (a) and the exon structure of the exon 3-deleted isoform (b) that would allow for the translation of a truncated form of preproghrelin with an alternative, novel C-terminal peptide sequence RPQPTSDRPQALLTSL (SEQ ID NO:1).

FIG. 3: (a) GHS-R 1a and (b) 1b and (c) ghrelin RT-PCR products amplified from the ALVA (A) DU145 (D) LNCaP (L) and PC3 (P) prostate cancer cell lines and normal prostate cDNA library (N). C1=no template negative control, C2=GHS-R 1b representative PCR performed on ALVA-41 RNA preparation prior to reverse transcription.

FIG. 4: Agarose gel stained with ethidium bromide showing specific RT-PCR products of the expected size generated using GHS-R 1b specific primers. The nature of these transcripts was confirmed using cDNA sequencing. 1-3. JAR choriocarcinoma cell line 4-5. normal term human placenta 6-8. JEG choriocarcinoma cell line 9. normal human stomach 10-12. Hec1a endometrial cancer cell line 13-14. Hec 1b human endometrial cancer cell line. 16. normal human prostate 17. negative control. M=molecular weight marker. GHSR 1b RT-PCRs were also positive for the MCF7, T47D, and MDA-MB 231 breast cancer cell lines, in the Ishikawa and KLE endometrial cancer cell lines, in the OvCar3 ovarian cancer cell line and in the ALVA-41, DU145, PC3 and LNCaP prostate cancer cell lines (data not shown).

FIG. 5: Ethidium bromide stained agarose gels showing RT-PCR products in prostate cancer cell lines and normal cDNA and female reproductive cancer cell lines and normal breast cDNA using primers specific for the exon 3-deleted isoform of preproghrelin. 1. Molecular weight marker 2. normal prostate cDNA 3. DU-145 4. LNCAP 5. PC-3 6. normal breast cDNA 7. MCF-7 8. T47D 9. Jar 10. LCCS 11. KLE 12. Hec 1b 13. Ishikawa 14 negative control.

FIG. 6: Immunohistochemistry performed on ALVA41 (a,f), DU145 (b,g), LNCAP (c,l) and PC3 (d,i) cells using GHS-R 1a antibodies (a-d) and ghrelin antibodies (f-i). Positive staining is indicated by the brown cytoplasmic staining. All cell nuclei are non-immunoreactive. Representative ghrelin negative control (e) demonstrates lack of immunoreactivity in LNCaP cells.

FIG. 7: Immunohistochemistry demonstrating strong immunoreactivity for GHS-R1b in prostate cancer glands (b) as opposed to negative staining in glands from normal prostate tissue (a).

FIG. 8: Immunohistochemical staining using GHSR 1b-specific antibody in (a) human breast tumour section and (b) normal breast tissue. Nest of cancer cells exhibiting strong cytoplasmic staining (arrow) and non-immunoreactive nuclei. L=lumen of a normal breast duct.

FIG. 9: Immunohistochemical staining of normal and histopathological prostate specimens using anti-ghrelin antibody. (a) Normal prostate tissue demonstrating weak epithelial (arrow) cytoplasmic immunostaining for ghrelin. (b) Prostate cancer glands demonstrating strong cytoplasmic immunoreactivity for ghrelin. E=epithelial cells, S=stroma, L=lumen, G=gland FIG. 10: Immunohistochemical staining of normal prostate and histopathological specimens using the exon 3-deleted ghrelin antibody. (a) Normal prostate tissue demonstrating weak epithelial (arrow) cytoplasmic immunostaining for exon 3-deleted ghrelin. (b) In contrast to (a), prostate cancer glandular epithelial cells (arrow) stain intensely for exon 3-deleted ghrelin. E=epithelial cells, S=stroma, G=gland, L=lumen.

FIG. 11: Western blot of cell extracts from the ALVA41, BPH-1 and DU145 prostate cancer cell lines using the GHSR1b specific antibody reveals a single band of 45 kda representing the 1b protein.

FIG. 12: Western immunoblot of cell lysates from LNCaP (L), PC3 (P), ALVA41 (A) and DU145 (D) prostate cancer cell lines (24 h exposure to X-ray film) using anti-human ghrelin antibody showing bands of approximately 3 KDa, identical to synthetic human n-octanoylated ghrelin (1 μg, lane G) which was exposed for a shorter time (5 min).

FIG. 13: The effect of ghrelin on PC3 cells. Cell proliferation was determined using the MTT dye method. Absorbance readings for each were converted to percentage above control±SEM, indicated by error bars. * denotes P values <0.01, ** P<0.001 (one-way ANOVA with Tukey's post hoc comparisons). Data represents one of 3 identical experiments, each n=16.

FIG. 14: The effect of ghrelin on MDA-MB231 and Ishikawa cells. Cell proliferation was determined using the MTT dye method. Absorbance readings for each were converted to percentages above control±SEM, indicated by error bars. * denotes P values <0.01, ** P<0.001 (one-way ANOVA with Tukey's post hoc comparisons).

### DETAILED DESCRIPTION OF THE INVENTION

The present invention arises from the discovery that ghrelin, GHS-R 1a and GHS-R 1b are expressed in prostate cancer cells. More particularly, ghrelin and GHS-R 1b protein and nucleic acid are expressed at higher levels in prostate cancer and breast cancer cells compared to the levels observed in normal prostate and normal breast. Another feature of the present invention is the discovery of a novel, exon 3-deleted form of preproghrelin the expression of which distinguishes prostate cancer and breast cancer cells from their normal counterparts.

The present invention therefore provides methods that allow cancer cells and tissues of the reproductive system to be distinguished from normal cells and tissues, and therapeutic methods and novel agents for treating cancer by inhibition or suppression of ghrelin activity. That is, by suppression or inhibition of ghrelin itself, GHS-R 1a or GHS-R 1b and homologous ligands or receptors that are associated with cancers of the reproductive system. The present inventors propose that these methods and novel agents may well be useful in treating cancers of the reproductive system where ghrelin is expressed, or more particularly in reproductive cancers where both ghrelin, GHS-R 1a and GHS-R 1b are co-expressed, given that GHS-R 1a is known to bind ghrelin.

Cancers of the reproductive system include, but are not limited to, prostate cancer, breast cancer, ovarian cancer, cervical cancer, choriocarcinoma and uterine cancer. With regard to disease treatment, the present invention also contemplates hyperproliferative disorders of the reproductive system such as benign prostatic hyperplasia.

Exon 3-Deleted Preproghrelin Protein and Nucleic Acid

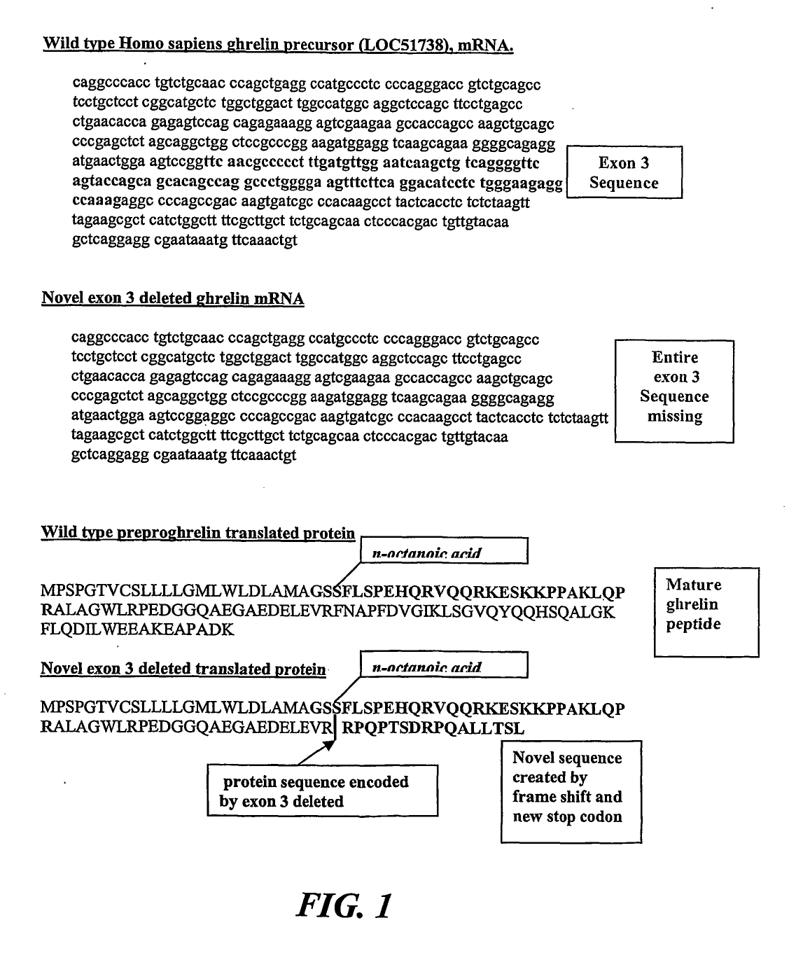

One aspect of the invention provides a novel isoform of an isolated preproghrelin protein that could be of functional significance in cancer and may be useful in cancer diagnosis and therapy. This novel form of preproghrelin is set forth in SEQ ID NO:2 (FIG. 1) and is characterized by a novel C-terminal peptide sequence RPQPTSDRPQALLTSL (SEQ ID NO: 1).

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native or recombinant form.

By “protein” is meant an amino acid polymer. The amino acids may be natural, non-natural D- or L-amino acids as are well understood in the art.

A “peptide” is a protein having no more than fifty (50) amino acids.

A “polypeptide” is a protein having fifty (50) or more amino acids.

The C-terminal sequence RPQPTSDRPQALLTSL (SEQ ID NO: 1) of exon 3-deleted preproghrelin is a novel peptide fragment that may be produced through the application of standard recombinant nucleic acid techniques or synthesized using conventional liquid or solid phase synthesis techniques. Alternatively, peptides can be produced by digestion of exon 3-deleted preproghrelin with proteinases such as trypsin or staphylococcus V8-protease. The digested fragment can be purified by, for example, high performance liquid chromatographic (HPLC).

Also provided are variants of exon 3-deleted preproghrelin in which one or more amino acids have been replaced by different amino acids or non-natural amino acids, for example. It is well understood in the art that some amino acids may be changed to others with broadly similar properties without changing the nature of the activity of the protein (conservative substitutions).

The invention also contemplates chemical modification of exon 3-deleted preproghrelin. These include, but are not limited to, chemical modification of side chains, incorporation of unnatural amino acids and/or their derivatives during protein synthesis, N- and O-linked glycosylation, fatty acylation (such as addition of N-octanoic acid), acetylation, oxidation of sulfhydryls, biotinylation, conjugation with fluorochromes, dyes and crosslinkers. A more detailed discussion of chemical modification of proteins is provided in Chapter 20 of CURRENT PROTOCOLS IN PROTEIN SCIENCE Eds. Coligan et al. (John Wiley & Sons NY, USA 1995-2001).

For the purposes of recombinant expression and purification of exon 3deleted preproghrelin, “fusion partners” may be employed, typically at the N- or C-terminus of the expressed protein together with an appropriate affinity matrix. Examples of fusion partners are glutathione-S-transferase (GST), Fc portion of human IgG, maltose binding protein (MBP) and hexahistidine (HIS₆), which are particularly useful for isolation of the fusion protein by affinity chromatography with glutathione-, Protein-A or -G, amylose-, and nickel- or cobalt-conjugated resins respectively. Many such matrices are available in “kit” form, such as the QIAexpress™ system (Qiagen) useful with (HIS₆) fusion partners and the Pharmacia GST purification system.

Fusion partners according to the invention also include within their scope “epitope tags”, which are usually short peptide sequences for which a specific antibody is available. Well known examples of epitope tags for which specific monoclonal antibodies are readily available include c-myc, influenza virus haemagglutinin and FLAG tags.

Recombinant protein expression and vectors suitable therefor are well known in the art such as described in Chapters 10 and 16 of CURRENT PROTOCOLS IN PROTEIN SCIENCE Eds. Coligan et al. (John Wiley & Sons NY USA 1995-2001).

Suitable host cells for recombinant expression may be prokaryotic or eukaryotic, such as Escherichia coli (DH5α, SURE and XL1-Blue for example), yeast cells, Sf9 cells utilized with a baculovirus expression system, CHO cells, COS, CV-1 and 293 cells, without limitation thereto.

The invention also provides an isolated nucleic acid that encodes said exon 3-deleted preproghrelin protein.

An embodiment of said nucleic acid has a nucleotide sequence as set forth in FIG. 1 (SEQ ID NO:3).

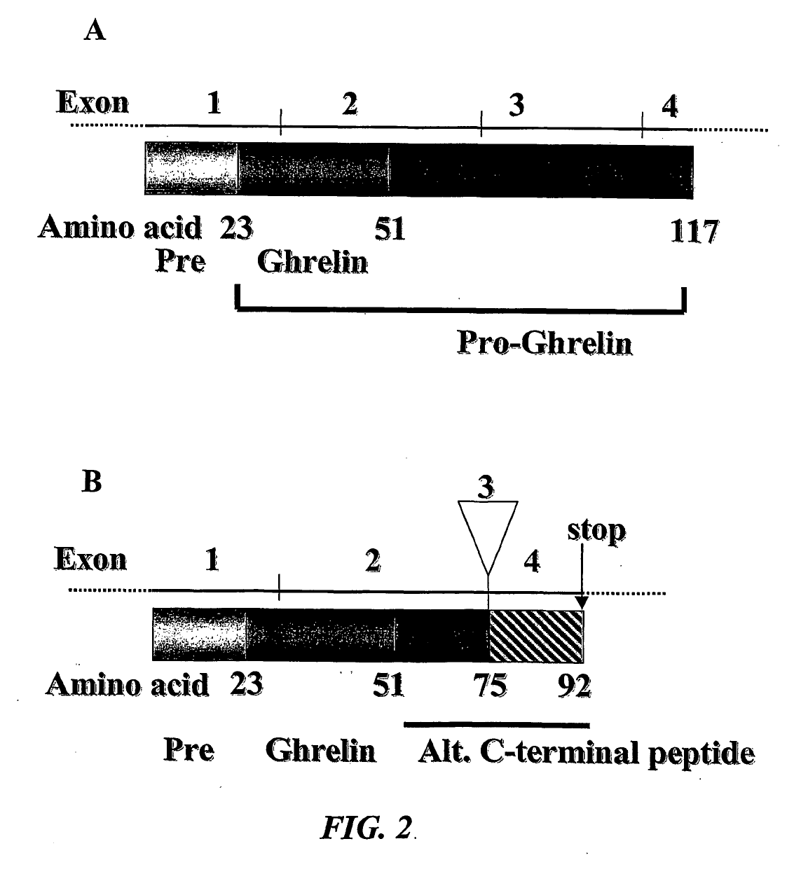

Referring to FIGS. 1 and 2, the mRNA encoding this isoform has a complete deletion of exon 3 of the pro- form of ghrelin, which was first described by Kojima et al., 1999, Nature 402 656. Translation of this isoform would lead to the production of wild-type, mature ghrelin coded by exon 1 and part of exon 2 (FIGS. 1 and 2). The deletion would lead to a frameshift, however, that would disrupt the original stop codon within exon 4 and would lead to the production of a novel C-terminal peptide sequence (RPQPTSDRPQALLTSL stop).

As used herein, “nucleic acid” encompasses single- or double-stranded mRNA, RNA, cRNA and DNA inclusive of cDNA and genomic DNA. A “polynucleotide” is a nucleic acid having eighty (80) or more contiguous nucleotides, while an “oligonucleotide” has up to eighty (80) contiguous nucleotides.

A “probe” may be a single or double-stranded oligonucleotide or polynucleotide, suitably labeled for the purpose of detecting complementary sequences in Northern or Southern hybridization, for example.

A “primer” is usually a single-stranded oligonucleotide, preferably having 15-50 contiguous nucleotides, which is capable of annealing to a complementary nucleic acid template and being extended in a template-dependent fashion by the action of a DNA polymerase such as Taq polymerase, RNA-dependent DNA polymerase or Sequenase™.

The terms “anneal”, “hybridize” and “hybridization” are used herein in relation to the formation of bimolecular complexes by base-pairing between complementary or partly-complementary nucleic acids in the sense commonly understood in the art. It should also be understood that these terms encompass base-pairing between modified purines and pyrimidines (for example, inosine, methylinosine and methyladenosine) and modified pyrimidines (for example thiouridine and methylcytosine) as well as between A,G,C,T and U purines and pyrimidines. Factors that influence hybridization such as “stringency” in terms of temperature, ionic strength, duration and denaturing agents are well understood in the art, although a useful operational discussion of hybridization is provided in Chapter 2 of CURRENT PROTOCOLS IN MOLECULAR BIOLOGY (Eds. Ausubel et al. John Wiley & Sons NY, 2000), particularly at sections 2.9 and 2.10.

Also within the scope of the invention are isolated nucleic acids encoding variants and derivatives of exon 3-deleted preproghrelin and proghrelin and proteins homologous thereto. Such isolated nucleic acids may be isolated, for example, by nucleic acid sequence amplification using degenerate primers or by hybridization with SEQ ID NO: 3 under appropriate stringency conditions as hereinbefore described.

Detection Methods

The present invention provides methods of detecting ghrelin, the exon 3deleted form of preproghrelin and/or GHS-R 1b expression by a cell or tissue of the reproductive system as an indicator that said cell or tissue of the reproductive system is cancerous. The data from prostate cancer cells suggest that ghrelin and/or GHS-R 1b expression may be particularly useful as diagnostic indicators of prostate cancer.

Also contemplated by the invention is detection of exon-3 deleted preproghrelin nucleic acid and protein.

As used herein, “reproductive system” includes and encompasses the male and female reproductive system including organs and tissues such as prostate, testis, breast, ovary, ovarian follicles, vagina, fallopian tubes, cervix and uterus.

In one embodiment, a nucleic acid-based detection method is performed.

Preferably, nucleic acid detection is performed by PCR analysis.

An example of PCR amplification is provided hereinafter, although the skilled person is referred to Chapter 15 of CURRENT PROTOCOLS IN MOLECULAR BIOLOGY (Eds. Ausubel et al. John Wiley & Sons NY 1995-2000) for a general discussion of PCR methodology.

PCR amplification may be combined with other methods such as Southern hybridization and nucleic acid sequencing to identify ghrelin and GHS-R 1b nucleic acids. These methods are well known to persons skilled in the art. Specific examples of Southern analysis and sequencing of PCR products are provided hereinafter in the Examples.

It will also be appreciated that nucleic acid sequence amplification techniques other than PCR may be useful according to the invention. Potentially suitable nucleic acid amplification techniques other than PCR are well known to the skilled addressee and include strand displacement amplification (SDA); rolling circle replication (RCR) as for example described in Liu et al., 1996, J. Am. Chem. Soc. 118 1587, International application WO 92/01813 and International Application WO 97/19193; nucleic acid sequence-based amplification (NASBA) as for example described by Sooknanan et al., 1994, Biotechniques 17 1077; ligase chain reaction (LCR) as for example described in International Application WO89/09385 and Chapter 15 of CURRENT PROTOCOLS IN MOLECULAR BIOLOGY supra; and Q-β replicase amplification as for example described by Tyagi et al. 1996, Proc. Natl. Acad. Sci. USA 93 5395.

As used herein, an “amplification product” refers to a nucleic acid product generated by any nucleic acid amplification technique.

It will also be well understood by the skilled person that detection of ghrelin, the exon 3-deleted form of preproghrlein and/or GHS-R 1b nucleic acids may be performed using any of a variety of techniques such as RNA detection, fluorescence-based melt curve analysis, nucleic acid arrays (e.g. microarrays) and other methods that utilize hybridization of nucleic acid probes.

RNA detection may be performed by methods such as Northern blotting, RNAse protection and primer extension as are well known in the art, although skilled persons are referred to Chapter 4 of CURRENT PROTOCOLS IN MOLECULAR BIOLOGY (Eds. Ausubel et al. John Wiley & Sons NY 1995-2000) for exemplary methods relating to RNA detection.

Melt curve analysis can be performed using fluorescent DNA-intercalating dyes to detect PCR product formation either as an end-product or in “real time”. Fluorochrome-labeled probes can also be used to detect formation of specific products either during or after completion of PCR. A useful example of melt curve analysis can be found, for example, in International Publication No. WO97/46714.

Microarrays also utilize hybridization-based technology that, for example, may allow allele detection by way of hybridization of a nucleic acid sample to ghrelin, exon 3-deleted preproghrelin and/or GHS-R 1b-specific probes immobilized on an appropriate substrate as is well understood in the art. In this regard, the skilled person is referred to Chapter 22 of CURRENT PROTOCOLS IN MOLECULAR BIOLOGY (Eds. Ausubel et al. John Wiley & Sons NY, 2000), International Publication WO00/58516, U.S. Pat. No. 5,677,195 and U.S. Pat. No. 5,445,934 which provide exemplary methods relating to nucleic acid array construction and use in detection of nucleic acids of interest.

In another embodiment, detection of ghrelin, exon 3-deleted preproghrelin and/or GHS-R 1b expression by reproductive cancer cells is performed by protein analysis according to methods well known in the art.

Suitable methods include ELISA, immunohistochemistry, immunoblotting, immunoprecipitation and any of a variety of chromatographic separation/identification methods without limitation thereto.

These methods may be assisted by use of labeled antibodies to ghrelin, exon 3-deleted preproghrelin and/or GHS-R 1b as will be discussed hereinafter, or by protein labeling techniques such as radiolabeling with ³⁵S, ¹⁴C or ¹²⁵I and biotinylation, as for example described in Chapter 2 of CURRENT PROTOCOLS IN PROTEIN SCIENCE (Eds. Coligan et al. John Wiley & Sons NY 1995-2000).

An example of an immunohistochemistry method is provided in detail hereinafter.

A preferred ELISA method utilizes a GHS-R 1b-specific polyclonal antibody produced as will be described hereinafter. Preferably, a sample is subjected to a “sandwich” ELISA where the GHS-R 1b-specific antibody is immobilized to an ELISA plate. GHS-R 1b protein in the fluid sample binds the immobilized antibody and non-bound material is washed away. A second antibody that recognizes both GHS-R 1a and 1b is then added to the ELISA plate. Detection of GHS-R 1b-containing complexes is then performed either by virtue of the second antibody being labeled, or by addition of a labeled tertiary antibody that specifically binds an unlabeled second antibody.

Examples of chromatographic separation, affinity purification and immunoprecipitation and immunoblotting methods are provided in Chapters 8, 9 and 10 of CURRENT PROTOCOLS IN PROTEIN SCIENCE supra, respectively.

Antibodies

The invention also provides an antibody capable of binding ghrelin, exon 3-deleted preproghrelin, GHS-R 1a or GHS-R 1b.

In one embodiment, said antibody is capable of inhibiting binding between ghrelin and said GHS-R 1a. However, non-neutralizing antibodies are also contemplated as being useful in detection methods as hereinbefore described.

Such antibodies may be polyclonal, obtained for example by immunizing an animal with ghrelin, GHS-R 1a, GHS-R 1b, exon 3-deleted preproghrelin or a fragment thereof.

In one embodiment, said antibody is capable of distinguishing between the aforementioned exon 3-deleted form of preproghrelin and preproghrelin.

Preferably, the antibody is capable of binding the amino acid sequence RPQPTSDRPQALLTSL, an antigenic fragment thereof and/or a protein comprising same.

Suitably, said animal could be a mouse, rat, rabbit, sheep, chicken or goat.

Preferably, the animal is a rabbit.

Alternatively, monoclonal antibodies may be produced by standard methods such as described in CURRENT PROTOCOLS IN IMMUNOLOGY (Eds. Coligan et al. John Wiley & Sons. 1995-2000) and Harlow, E. & Lane, D. Antibodies: A Laboratory Manual (Cold Spring Harbour, Cold Spring Harbour Laboratory, 1988). Such methods generally involve obtaining antibody-producing cells, such as spleen cells, from an animal immunized as described above, and immortalizing said cell, such as by fusion with an immortalized fusion partner cell.

As is well understood in the art, antibodies maybe conjugated with labels selected from a group including a chromogen, a catalyst, an enzyme, a fluorophore, a chemiluminescent molecule, biotin and a radioisotope.

A large number of enzymes suitable for use as labels is disclosed in United States Patent Specifications U.S. Pat. No. 4,366,241, U.S. Pat. No. 4,843,000, and U.S. Pat. No. 4,849,338, each of which is herein incorporated by reference. Suitable enzyme labels useful in the present invention include alkaline phosphatase, horseradish peroxidase, luciferase, β-galactosidase, glucose oxidase, lysozyme, malate dehydrogenase and the like. The enzyme label may be used alone or in combination with a second enzyme in solution.

Fluorophores may be selected from a group including fluorescein isothiocyanate (FITC), tetramethylrhodamine isothiocyanate (TRITC), allophycocyanin (APC), Texas Red (TR), Cy5 or R-Phycoerythrin (RPE). Examples of useful fluorophores may be found, for example, in U.S. Pat. No. 4,520,110 and U.S. Pat. No. 4,542,104 which are herein incorporated by reference.

For the purposes of a diagnostic test that detects GHS-R 1b (such as an ELISA), a particular anti-GHS-R 1b antibody is contemplated. GHS-R 1a and 1b proteins have common domains and to date there has been no antibody described that can distinguish GHS-R 1b from GHS-R 1a. GHS-R 1a is a 366 amino acid peptide with 7 transmembrane domains. The GHS-R 1b mRNA arises from alternative splicing of the gene encoding GHS-R 1a. This mRNA encodes a 289 amino acid peptide that lacks the 6th and 7th transmembrane domains. The GHS-R 1b mRNA incorporates additional intronic sequence that encodes a 24 amino acid sequence that would be a useful target for GHS-R 1b-specific antibodies. The present inventors have designed a peptide: H-GGSQRALRLSLAGPILSLC-NH2, based on the amino acid sequence disclosed in Howard et al., 1996, Science 273 974). This peptide has been conjugated to a carrier, injected into rabbits and polyclonal antiserum raised. Following affinity purification, this antibody was used for detection of GHS-R 1b as hereinafter described in detail.

Ghrelin Antagonists

The present invention contemplates antagonists that suppress or inhibit the ghrelin system, and use of such agents in therapy of cancers of the reproductive system. Such antagonists may disrupt or prevent binding of ghrelin to GHS-R 1a or GHS-R 1b, for example.

Although not wishing to be bound by theory, the present inventors propose that expression of ghrelin, GHS-R 1a and GHS-R 1b by prostate cancer cells may constitute a paracrine/autocrine loop whereby unchecked cancer cell proliferation is maintained. Inhibition of this cell proliferation by targeting the ghrelin system (inclusive of ghrelin, GHS-R 1a and GHS-R 1b and related or homologous ligands and receptors) is contemplated by the present invention.

Because ghrelin is known to bind GHS-R 1a, this is the preferred target for interventionist strategies aimed at suppressing ghrelin activity and thereby treating cancers of the reproductive system.

Suppression or inhibition of ghrelin activity can readily be monitored at the cellular level by measuring cancer cell proliferation (such as by BrdU incorporation), in vitro cancer cell invasion and motility (as for example described in Leavesley et al., 1993, J. Cell. Biol. 121 163; Melchiori et al., 1992, Cancer Res. 52 2353) and by detecting cell apoptosis.

Such methods are applicable to reproductive cancers including prostate cancer, breast cancer, ovarian cancer and cervical cancer, without limitation thereto.

To this effect, neutralizing antibodies that disrupt ghrelin binding by GHS-R1 a constitute an embodiment of an antagonist according to the invention.

Other antagonists may be “mimetics” that mimic the binding interaction between ghrelin and GHS-R 1a or GHS-R 1b and thereby block ghrelin binding. These may be peptides, polypeptides or other organic molecules, preferably small organic molecules, with a desired biological activity and half-life.

One particular antagonist contemplated by the present invention is a non-n-octanoylated form of ghrelin which is known to be inactive in terms of stimulating GH secretion. N-octanoylation is a natural post-translational modification of ghrelin that appears to be necessary for the function of this ligand.

Other examples of antagonists contemplated by the present invention are growth hormone releasing peptide (GHRP) antagonists such as dynorphin A and des-tyr-dynorphin (Codd et al., 1990, Neuropeptides 15 133) and L-756,867 (Cheng et al., 1997, J. Endocrinol 152 155). Substance P antagonists [DArg1DTrp7,9Leu11]SP (P-7482) and [DArg1-DPro2DTrp7,9Leu11]SP(P-7483) (Bitar et al., 1991, Biochem. Biophys. Res. Comm. 15 156) may also be employed as ghrelin antagonists and are commercially available from Sigma (catalogue numbers S3641 and S4152).

A further example of a ghrelin antagonist is a substance P antagonist also known as Antagonist D and also commercially available from Sigma (catalogue number S3144; Cheng et al., 1997, J. Endocrinol. 152 155).

It should also be appreciated that there are a number of other substance P antagonists that could be readily assayed for antagonism of the ghrelin system by methods such as those described above. In this regard the skilled person is referred to Caranikas et al., 1982, J. Med. Chem. 25 1313, Lundberg et al., 1983, Proc. Natl. Acad. Sci. USA 80 1120, Engberg et al., 1981, Nature 293 222 Mizrahi et al., 1982, Eur. J. Pharmacol. 82 101 and Leander, 1981, Nature 294 467 for examples of substance P antagonists that might be useful as antagonists of the ghrelin system.

It is also noted that anti-ghrelin immunoglobulin G inhibits ghrelin activity (Nakazato et al., 2001, Nature 409 194) and that GH release can be inhibited by a chimeric peptide consisting of GHRP-6 and somatostatin (Dasgupta et al., 1999, Biochem. Biophys. Res. Comm. 259 379). These are also candidate antagonists contemplated by the present invention.

It is envisaged by the present inventors that substance P antagonists such as those described above may be useful as antagonists in their own right, or as starting points for developing antagonists that negatively influence ghrelin activity in cancer cells and tissues of the reproductive system.

However, it should be appreciated that the present invention is not limited to use of the aforementioned antagonists. There are a variety of other ways that ghrelin system antagonists may be identified.

Mutagenesis of ghrelin is contemplated as being a potentially useful way of producing an antagonist of the invention. This can be performed by mutagenizing ghrelin protein or by mutagenizing an encoding nucleic acid, such as by random mutagenesis or site-directed mutagenesis. Examples of nucleic acid mutagenesis methods are provided in Chapter 9 of CURRENT PROTOCOLS IN MOLECULAR BIOLOGY, Ausubel et al., supra.

Both site-directed and random mutagenesis are well known in the art, and mutagenesis kits are commercially available, such as the Diversify™ random mutagenesis kit (Clontech).

Mutagenesis methods include chemical modification of proteins by hydroxylamine (Ruan et al., 1997, Gene 188 35), incorporation of dNTP analogs into nucleic acids (Zaccolo et al., 1996, J. Mol. Biol. 255 589) and PCR-based random mutagenesis such as described in Stemmer, 1994, Proc. Natl. Acad. Sci. USA 91 10747 or Shafikhani et al., 1997, Biotechniques 23 304, each of which references is incorporated herein.

Computer-assisted structural database searching is becoming increasingly utilized as a procedure for identifying mimetics. Typically, these methods create space-filling models of a ligand (such as ghrelin) and receptor (GHS-R 1a, for example) interaction so as to search for candidate structures that may interrupt this binding interaction. Database searching methods which, in principle, could be suitable for identifying mimetics, may be found in International Publication WO 94/18232 (directed to producing HIV antigen mimetics), U.S. Pat. No. 5,752,019 and International Publication WO 97/41526 (directed to identifying EPO mimetics), each of which is incorporated herein by reference. Alternatively, computer-assisted molecular modelling is used to rationally design molecules that may interrupt the ghrelin GHS-R 1b binding interaction.

Other methods include a variety of biophysical techniques which identify molecular interactions, such as GHS-R 1a or GHS-R 1b receptor/ligand binding events. These may allow for the screening of candidate molecules according to whether said candidate molecule affects binding between ghrelin and GHS-R 1a, for example. Methods applicable to potentially useful techniques such as competitive radioligand binding assays, analytical ultracentrifugation, microcalorimetry, surface plasmon resonance and optical biosensor-based methods are provided in Chapter 20 of CURRENT PROTOCOLS IN PROTEIN SCIENCE Eds. Coligan et al., (John Wiley & Sons, 1997) which is incorporated herein by reference.

The present invention also contemplates isolation of antagonists by way of screening libraries of molecules such as synthetic chemical libraries, including combinatorial libraries, by methods such as described in Nestler & Liu, 1998, Comb. Chem. High Throughput Screen. 1 113 and Kirkpatrick et al., 1999, Comb. Chem. High Throughput Screen 2 211.

It is also contemplated that libraries of naturally-occurring molecules may be screened by methodology such as reviewed in Kolb, 1998, Prog. Drug. Res. 51 185.

Pharmaceutical Compositions

The invention includes pharmaceutical compositions comprising agents that suppress or inhibit ghrelin activity. Suitably, the pharmaceutical composition comprises a pharmaceutically-acceptable carrier.

By “pharmaceutically-acceptable carrier” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline, and pyrogen-free water.

Any suitable route of administration may be employed for providing a patient with the composition of the invention. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Pharmaceutical compositions of the present invention suitable for oral or parenteral administration may be presented as discrete units such as capsules, sachets or tablets each containing a pre-determined amount of one or more therapeutic agents of the invention, as a powder or granules or as a solution or a suspension in an aqueous liquid, a non-aqueous liquid, an oil-in-water emulsion or a water-in-oil liquid emulsion.

Therapeutic Methods

The present invention provides methods and agents for treating cancers of the reproductive system, preferably prostate cancer or breast cancer.

Such methods may utilize ghrelin, GHS-R 1a and/or GHS-R 1b antagonists as hereinbefore described, preferably in the form of appropriate pharmaceutical compositions.

Also contemplated by the present invention are methods and agents utilizing nucleic acids that encode ghrelin, GHS-R 1a and/or GHS-R 1b.

More particularly, gene therapy methods are contemplated whereby a ghrelin, GHS-R 1a or GHS-R 1b nucleic acid are oriented in an antisense (3′ 5′) orientation in an expression vector suitable for administration to mammals such as humans.

An “expression vector” is a nucleic acid comprising appropriate regulatory sequences that direct expression of a nucleic acid operably linked thereto. Expression vectors may either be a self-replicating extra-chromosomal vector or a vector that integrates into a host genome.

By “operably linked” is meant that said regulatory nucleotide sequence(s) is/are positioned relative to the nucleic acid to be expressed to thereby initiate, regulate or otherwise control transcription.

Regulatory nucleotide sequences will generally be appropriate for the host cell used for expression. Numerous types of appropriate expression vectors and suitable regulatory sequences are known in the art for a variety of host cells.

Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences.

Constitutive or inducible promoters as known in the art are contemplated by the invention. The promoters may be either naturally occurring promoters, or hybrid promoters that combine elements of more than one promoter.

In a preferred embodiment, the expression vector contains a selectable marker gene to allow the selection of transformed host cells. Selectable marker genes are well known in the art and will vary with the host cell used.

The term “expression vector” also includes within its scope vectors generally known as “gene therapy vectors” such as vaccinia, and viral vectors useful in gene therapy. The latter include adenovirus and adenovirus-associated viruses (AAV) such as described in Braun-Falco et al., 1999, Gene Ther. 6 432, retroviral and lentiviral vectors such as described in Buchshacher et al., 2000, Blood 95 2499 and vectors derived from herpes simplex virus and cytomegalovirus. A general overview of viral vectors useful in endocrine gene therapy is provided in Stone et al., 2000, J. Endocrinol. 164 103.

Administration of the gene therapy construct to an animal, preferably a human individual, may include delivery via direct oral intake, systemic injection, or delivery to selected tissue(s) or cells, or indirectly via delivery to cells isolated from the mammal or a compatible donor. An example of the latter approach would be stem-cell therapy, wherein isolated stem cells having potential for growth and differentiation are transfected with a gene therapy construct which includes, for example, a ghrelin, GHS-R 1a or GHS-R 1b antisense nucleic acid. The stem cells are cultured for a period and then transferred to the animal being treated.

Delivery of said gene therapy construct to cells or tissues of said mammal or said compatible donor may be facilitated by microprojectile bombardment, liposome mediated transfection (e.g. lipofectin or lipofectamine), electroporation, calcium phosphate or DEAE-dextran-mediated transfection, for example. A discussion of suitable delivery methods may be found in Chapter 9 of CURRENT PROTOCOLS IN MOLECULAR BIOLOGY supra, for example.

So that the invention may be readily understood and put into practical effect, the skilled person is referred to the following non-limiting examples.

### EXAMPLE 1

### Materials and Methods

Cell Culture

ALVA41 cells were obtained from Dr P Leedman (Royal Perth Hospital, Perth, Australia) and DU145, LNCaP, and PC3 cells from the American Type Culture Collection (Rockville, Md.). ALVA41 cells were cultured in RPMI 1640 medium (pH 7.4) (Life Technologies, Rockville Md.) with 5% foetal calf serum (FCS) (CSL Biosciences, Melbourne, Australia) and DU145, LNCaP and PC3 cells in RPMI 1640 10% FCS. MCF7, MDAMB231, T47D breast cancer cell lines, KLE, Ishikawa, Hec 1A and Hec 1B endometrial cancer cell lines, JEG and JAR choriocarcinoma cell lines and the OvCar3 ovarian cancer cell line were cultured in DMEM/F12 (Life Technologies) with 10% FCS.

All cell lines were free from Mycoplasma and were cultured in 80 cm³ cell culture flasks (Nagle Nunc International, Roskilde, Denmark) at 37° C., 5% CO₂ with 50 units/ml penicillin G and 50 μl/ml streptomycin sulphate.

Reverse Transcriptase PCR (RT-PCR)

On reaching 70% confluence, total RNA was extracted from cell pellets with Trizol (Life Technologies) according to the manufacturer's instructions. RNA was incubated in a 50 μl solution containing 20 nM MgCl₂, 2 mM DTT, 0.5 mg Dnase (Rnase free, Roche, Basel Switzerland), 5.0 units of Rnase Inhibitor (Roche) and incubated for 30 min at 37° C. then heated at 90° C. for 5 min. Reverse transcription was achieved by the addition of 0.5 μg oligo dT₁₈ primer, 5 μg total RNA at 70° C. for 10 min. This solution was incubated at 43° C. for 2 min in buffer (50 mM Tris Cl pH 8.8, 75 mM KCl, 3 mM MgCl₂), 10mM DTT and 500 μl of each dNTP (pH 7; Roche). This was incubated with 200 units SuperScript RT II (Life Technologies) at 43° C. for 90 min, then 70° C. for 15 min.

A normal human prostate cDNA library was also obtained from Clontech, Palo Alto Calif. Normal human breast, ovarian, prostate and placental mRNA was also purchased from Clontech. PCR using β-actin primers—sense primer 5′GTGGGGCGCCCCAGGCACCA3′ (SEQ ID NO:6); antisense primer 5′TTGGCCTTGGGGTTCAGGGG3′ (annealing temp: 50° C.; SEQ ID NO:7) demonstrated the absence of genomic DNA contamination (330 bp product) in all cDNA samples. PCR was performed for GHS-R (1a and 1b) using sense primer 5′TCTTCCTTCCTGTCTTCTGTC3′ (SEQ ID NO:8) and antisense primers 5′AAGTCTGAACACTGCCACC3′ (type 1a, annealing temp: 50° C.; SEQ ID NO:9) and 5′CCTTCTCCCTTCTCTCTGA3′ (type 1b, annealing temp: 58° C.; SEQ ID NO: 10) and for ghrelin using sense primer 5′GAGGATGAACTGGAAGTCCG3′ (SEQ ID NO:11) and antisense primer 5′CATTTATTCGCCTCCTGAGC3′ (annealing temp: 59° C.; SEQ ID NO:12). PCRs contained 10×PCR Buffer, 100 μM dNTPs, 100 μM primers (Genset Pacific Oligos, Armidale, Australia), 2 μl cDNA or water (no template negative control) and 1 unit Red Hot Polymerase (Integrated Sciences, Melbourne, Australia). An additional “minus RT” negative control was performed with the GHS-R 1b PCRs for all cell lines. Thermal cycling consisted of 5 min at 95° C., 40 cycles of 30s, 95° C., 30s at annealing temperature, 2 min at 72° C., followed by 10 min at 72° C. on a PTC-200 Thermal cycler (MJ research, Watertown Mass.).

Southern Analysis and Sequencing

RT-PCR products electrophoresed on a 2% gel were capillary blotted overnight in 20×Standard saline citrate (3M NaCl, 0.3M sodium citrate pH 7) onto Hybond positively charged membranes (Amersham Pharmacia Biotech, Little Chalfont, UK). Internal oligonucleotide probes for GHS-R 5‘TGATGGCAGCACTGAGGTAG’ (SEQ ID NO:13) and ghrelin 5′TTGAACCGGACTTCCAGTTC3′ (Genset Pacific Oligos; SEQ ID NO:14) were labelled using a DIG dUTP/dATP tailing kit (Roche) and quantified and hybridised according to the DIG user's manual (Roche). Membranes were exposed to X-ray film (Agfa-Gavaert, Morstel, Belgium) for up to 15 min and developed using the Curix 60 automatic processor (Agfa-Gavaert). RT-PCR products were purified from an agarose gel using the Consert Rapid Gel PCR purification kit (Life Technologies). Sequencing was performed at the Australian Genome Research Facility (University of Queensland) using the Applied Biosystems 377 DN automated DNA sequencer and ABI Big Dye Terminator reagents.

Immunohistochemistry

Immunohistochemistry was performed using cultured cell lines and both normal and cancer histological sections (breast, prostate, endometrium, placenta, ovary). Immunohistochemistry was performed using our antibodies to GHSR1a, 1b, ghrelin and the exon 3-deleted form of proghrelin.

Cells were grown to 70% confluence in 96 well plates (Nagle-Nunc), washed in phosphate buffered saline (PBS) and fixed for 5 min in 100% methanol. Immunodetection was performed using a Histostain-SP plus broad spectrum diaminobenzamine staining kit (Zymed, San Franscisco, Calif.), according to the manufacturers instructions. Polyclonal anti-GHS-R 1a primary antibodies were raised in rabbits (IMVS, Adelaide, SA) against the C-terminal peptide fragment (RAWTESSINTC; SEQ ID NO:17) (Feighner et al., 1998, Mol. Endocrinol. 12 137) of the GHS-R 1a peptide and conjugated to diptheria toxin (Mimotopes, Victoria, Australia). Anti-ghrelin antibodies were raised against the whole human ghrelin peptide. Serum was diluted 1:100-1:3200 in 1% bovine serum albumin in 0.01M PBS, and cells were incubated at 4° C. for 24h. Negative controls included the abolition of staining by pre-absorbing the primary antibody with 1.0 mg/ml GHS-R 1a peptide fragment or with ghrelin or the omission of primary antibody.

Immunohistochemistry was performed on human sections using an Envision Plus DAB anti-rabbit immunostaining kit (Dako, USA) according to the manufacturer's instructions. Negative controls included the substitution of immune serum with 0.01M PBS 1% BSA and also preabsorption of the antibody with 1 mg/ml peptide overnight at 4° C. Positive controls consisted of pituitary sections for GHSR 1a and 1b (Peterborough) and human stomach for ghrelin (Peterborough). Sections were counterstained with haematoxylin.

Western Analysis

Western analysis was undertaken to confirm the expression of ghrelin peptide by the cells. Cell pellets were lysed in buffer containing 1% triton-X 100 and protease inhibitors (Roche). The homogenates were centrifuged and the supernatants containing total cell protein collected. 200 μg of protein was boiled for 2-3 min in 4×loading buffer (250 mM Tris-Cl pH 6.8, 2% SDS, 10% glycerol, 20 mM DTT, 0.01% bromophenol blue) and then electrophoresed, along with a Rainbow protein marker (Amersham) and 1 μg of synthetic ghrelin (Mimotopes) on a 10% SDS-PAGE gel. The protein was then transferred to a nitrocellulose membrane (Protran, Schleicher and Schuell, Germany) for 1 h in transfer buffer (10mM NaHCO₃, 3 mM Na₂CO₃, methanol). The membrane was blocked overnight at 4° C. in 1×Tris Buffered Saline/0.05% Tween 20/1% BSA. This was followed by incubation of the membrane in primary anti-ghrelin antibody or anti-GHSR1b antibody solution at 4° C. overnight. After washing in TBS/Tween20, the membrane was incubated with an anti-rabbit secondary antibody (1:1000 dilution) (DAKO, Carpintera, Calif.) at room temperature for 1 h. After washing, a 1:10 dilution of Femto chemiluminescence solution (Pierce, Rockford, Ill.) was layered onto the membrane and incubated for 5 min. The membrane was then exposed to X-ray film overnight and then developed using the Curix 60 automatic processor (Agfa-Gavaert).

Cell Proliferation Assays

PC3, MDAMB231 and Ishikawa cells were cultured in 96 well plates for 3 days at 37° C. in the presence of ghrelin (0-20 nM), in 10% FCS. Negative controls received no treatment. After aspiration of medium, cells were incubated in MTT (Sigma) solution (0.5 mg/ml) for 2h. MTT solution was aspirated and formazan salts solubilised in dimethyl sulfoxide (ICN, Costa Mesa, Calif.). A Biomeck Plate reader (Beckman, QLD, Australia) was used to measure absorbances (550/650nm).

Detection of Exon 3 Deleted Isoform of Preproghrelin Nucleic Acid

A PCR has been designed to specifically amplify the exon 3 deleted form of preproghrelin. The sense sequence (which spans exons 2 and 4) is 5′AATGGAGTCCGGAGGCCC-3′ (SEQ ID NO:15) and the antisense sequence 5′-GAACATTTATTCGCCTCCTG-3′ (SEQ ID NO:16). The structure of this novel exon 3-deleted form of preproghrelin is described in FIGS. 1 and 2.

Detection of Exon 3 Deleted Isoform of Preproghrelin Protein

A novel polyclonal antibody has been raised against the peptide RPQPTSDRPQALLTSL (SEQ ID NO:1). This peptide was conjugated to a carrier, injected into rabbits and polyclonal antiserum raised. The antibody was then affinity purified. This isoform of preproghrelin could therefore be specifically detected immunologically using Western blotting, ELISA or immunohistochemistry using this antibody.

Detection of GHS-1b Receptor Protein by ELISA

The LNCaP prostate cancer cell line and the benign prostatic hyperplasia cell line (BPH-1) were cultured in vitro to 70% confluency in 96 well plates (Nagle Nunc). BPH-1 and LNCaP cells were grown in RPMI 1640 with 10% foetal calf serum, containing 50 units/ml penicillin G and 50 μl/ml streptomycin sulphate (CSL Biosciences) and incubated at 37° C. in 5% CO₂ and 95% air. In addition BPH-1 cells were grown in the presence of ITS supplement (Sigma) and 20 ng/ml dihydrotestosterone. Cells were washed in phosphate buffered saline (PBS) and fixed for 5 min in ice-cold methanol. The methanol was removed and the cells allowed to air dry and were stored at −20° C. Cells were thawed in 50% methanol with 1% hydrogen peroxide and incubated in 2 changes of this solution for 20 min each. The cells were washed 3×5mins in ELISA wash buffer (0.01M PBS, 0.05% Tween) and blocked in 0.0-1M PBS with 1% bovine serum albumin (BSA) for 2 hours. After 3×5 min washed in ELISA wash buffer the cells were incubated in primary GHSR 1b antibody (as described previously) at dilutions of 12.5-100% in 0.01 M PBS, 1% BSA at 4° C. overnight. Negative control wells were treated with 0.01M PBS and 1% BSA without antibody. The antibody removed and the wells washed in ELISA wash buffer. Secondary antibody (Jackson Donkey anti-rabbit HRP conjugated IgG) was diluted 1/7500 in 0.01M PBS 1% BSA for 2 hours at room temperature. OPD with H₂O₂ urea buffer (Sigma, SigmaFast o-phenylenediamine dihydroxychloride tablet set) diluted in water (according to the manufacturer's instructions) was incubated with the cells for 2 hours at room temperature, protected from light. The reaction was stopped with 2.5M HCl and the absorbance read at 490 nm. Statistics were performed using Student's t-test and significance was considered to be a P value smaller than 0.05.

### EXAMPLE 2

### Results

RT-PCR Analysis

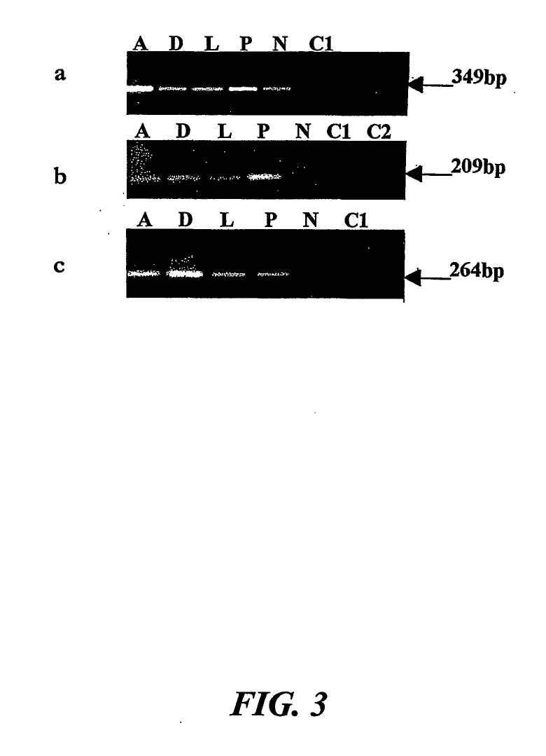

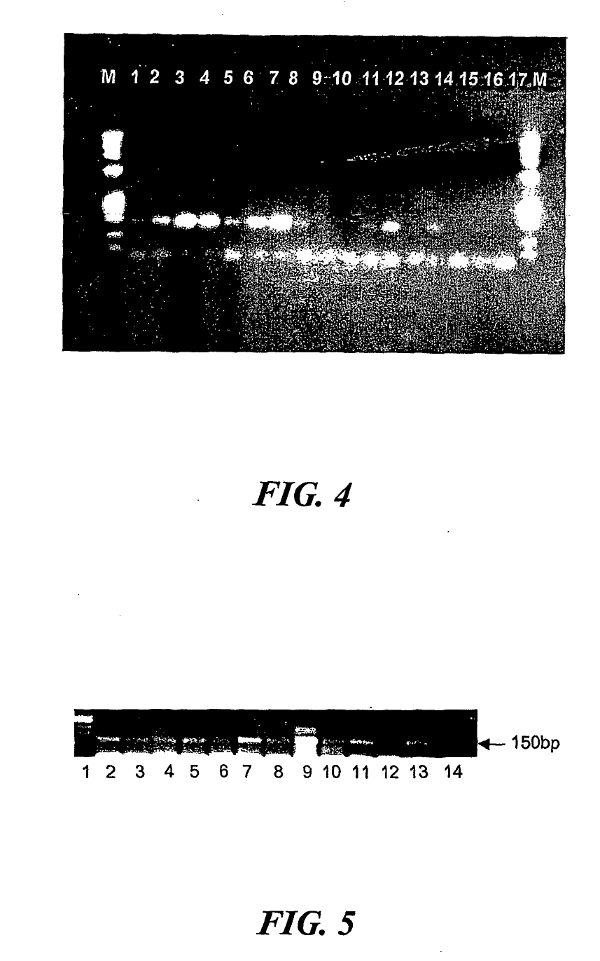

Single RT-PCR products of the expected size for GHS-R 1a (349 bp), GHS-R 1b (209 bp) and ghrelin (264 bp) were generated from cDNA derived from the ALVA41, DU145, LNCaP and PC3 prostate cancer cell lines (FIG. 3). Southern analysis of these blotted products detected signals corresponding to the expected size (data not shown) and their identity was confirmed by automated sequencing with all products 100% homologous to published sequences. Normal prostate cDNA library expressed the GHS-R 1a mRNA isoform but not GHS-R 1b isoform nor ghrelin. In addition to the β-actin screen (data not shown), RNA was Dnase-treated prior to reverse transcription to ensure that GHS-R 1b RT-PCR products were derived from cDNA and not genomic DNA, as the 3′ end of the GHS-R 1b mRNA transcript contains a short intronic sequence (Howard et al., 1996, supra). GHS-R 1b transcripts were also detected in cDNA derived from the JAR, placenta, JEG, Hec 1a, Hec 1b, MCF-7, T47D, MDA-MB 231, Ishikawa, KLE and OvCar3 cell lines (FIG. 4).

Using the primers hereinbefore described, PCR analysis demonstrated that the exon 3-deleted isoform of preproghrelin is present in the ALVA-41, DU145, LNCaP and PC3 prostate cancer cell lines and in normal prostate (data not shown). The exon 3 deletion has been demonstrated in the MCF7, MDA 231, T47D breast cancer cell lines, the Hec 1a, Hec 1a, KLE and Ishikawa endometrial cancer cell lines, and in the JAR choriocarcinoma cell line (many of these are shown in FIG. 5). It is absent or expressed at very low levels in normal breast.

Immunohistochemical Analysis

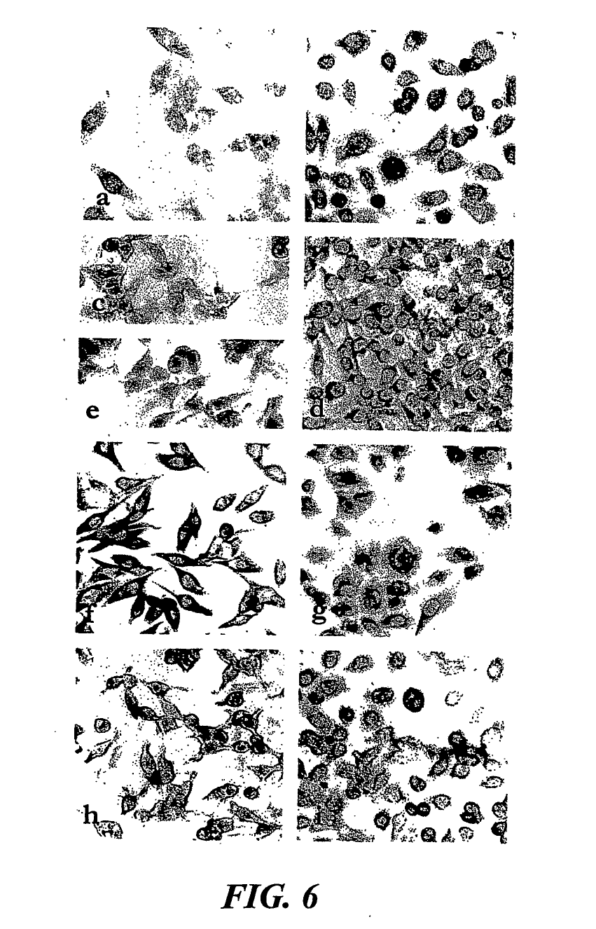

The results of all immunohistochemical analyses are provided in FIGS. 6, 7, 8, 9 and 10. A summary of immunohistochemical staining results is provided in Table 1.

Positive immunohistochemical staining for GHS-R 1a, GHS-R 1b, ghrelin and exon 3-deleted preproghrelin was present in the cytoplasm of all of the cell lines tested, providing evidence that these cells synthesise the GHS-R isoforms, ghrelin and exon 3-deleted preproghrelin proteins. Data for GHS-R 1a and ghrein are shown in respect of ALVA-41, DU 145, PC3 and LNCaP prostate cancer cells in FIG. 6. Primary antibody-free negative controls (FIG. 6e) and pre-absorption controls (data not shown) failed to stain for either antibody.

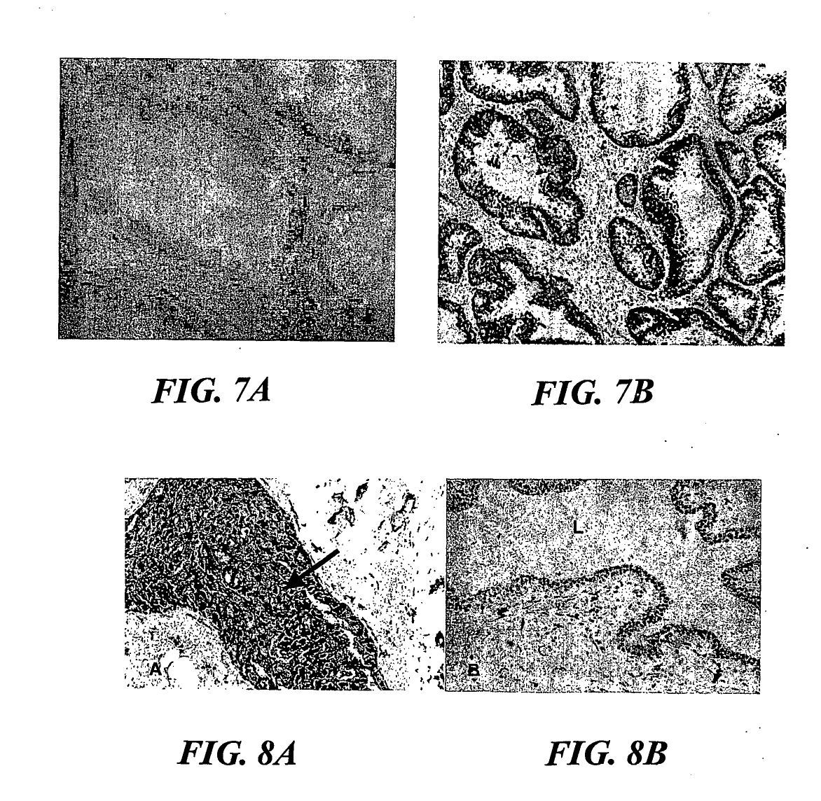

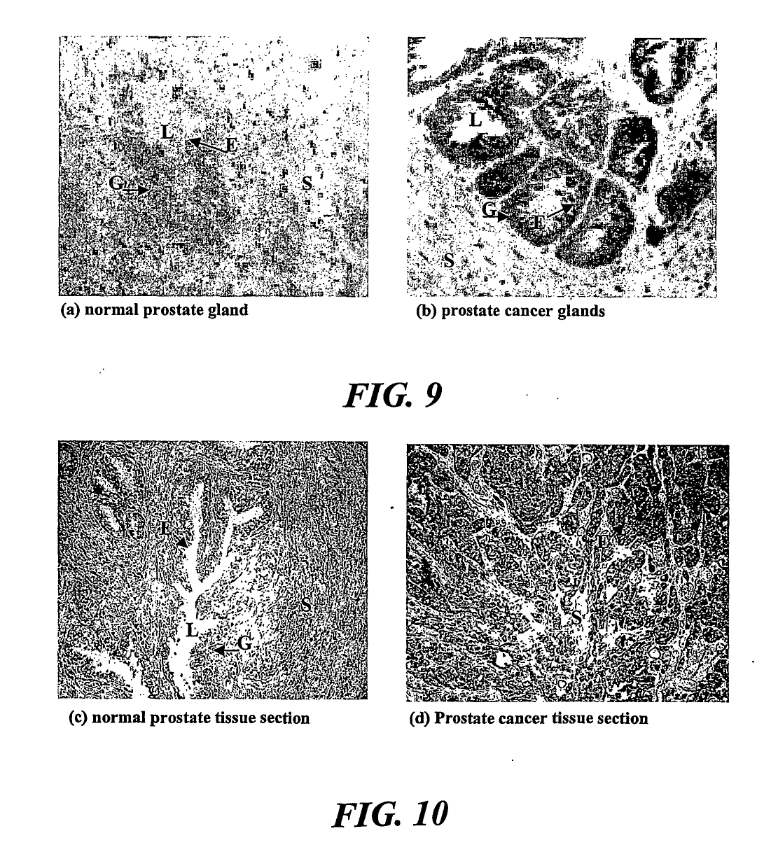

Immunohistochemistry performed on paraffin-embedded, archival tissue sections demonstrated that GHS-R 1a, GHS-R 1b, ghrelin and exon 3-deleted preproghrelin were all found to be expressed in prostate cancer tissues at the protein level. GHS-R 1a was found to be equally expressed in normal and cancer tissue (data not shown). GHS-R 1b protein was found to be expressed in the glandular epithelium of prostate cancer tissues but not in the normal prostate glands (FIG. 7). GHS-R 1b protein expression was also found in breast cancer glands but not in normal breast tissue (FIG. 8). Normal prostate tissue demonstrated weak immunoreactivity for ghrelin and exon 3-deleted preproghrelin protein in the epithelial cells of glands. Comparatively strong immunoreactivity for ghrelin and exon 3-deleted ghrelin was evident in the prostate tumour glands (FIGS. 9 and 10). All nuclei were non-immunoreactive in every tissue section. Primary antibody-free negative controls and pre-absorption controls failed to stain for any of the above antibodies (data not shown).

Western Blotting and ELISA Analysis

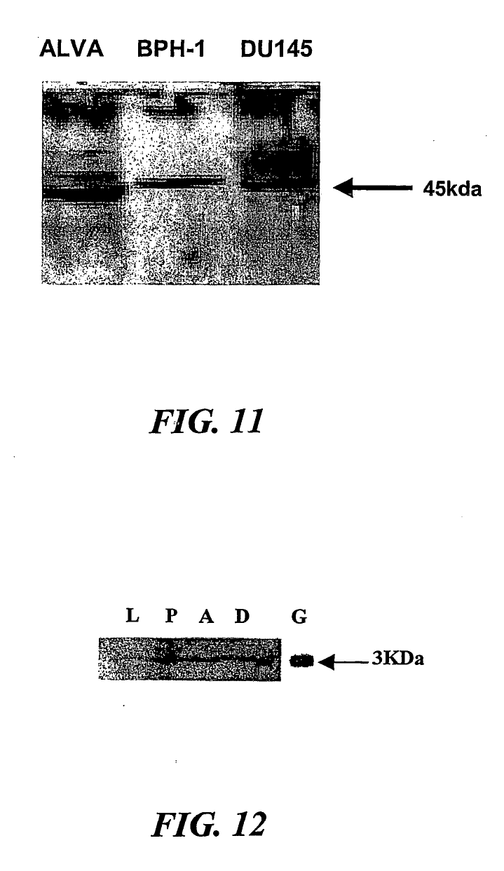

Western analysis on protein extracted from cell pellets, and using the same antibodies as for immunohistochemistry, demonstrated the expression of GHS-R 1a protein in ALVA-41, DU-145 and BPH-1 cell lines (data not shown). GHS-R 1b protein expression was demonstrated in ALVA-41, DU-145 and BPH-1 cell lines (FIG. 11). Ghrelin expression was detected in ALVA-41, DU-145, LNCaP and PC3 cell lines (FIG. 12). Exon 3-deleted proghrelin protein expression was demonstrated in the PC3 and DU-145 cell lines (data not shown). Western blots are therefore a useful method for detecting GHS-R 1b, ghrelin and exon 3-deleted proghrelin proteins and may be easily adapted for use as a quantitative assay.

Using an ELISA-based approach in intact, fixed LNCaP cells, absorbance at 490 nm of cells incubated with varying doses of anti GHS-R 1b antibody were dose dependent and significantly higher at the 2 highest doses (P<0.0037 and <0.016, respectively) than the non-antibody incubated control wells (n=5). In the BPH-1 cell line, absorbances were significantly greater (P<0.05) in antibody-treated than control wells across the whole range (12.5%-100%) of antibody dilutions made (n=2) (data not shown). The ELISA format is therefore useful for detecting GHS-R 1b protein expression directly on prostate cancer cells and may be easily adapted to become a quantitative assay for GHS-R 1b. Similar assays can be developed for ghrelin and/or exon 3-deleted preproghrelin expression.

Effect of Ghrelin upon cell Proliferation

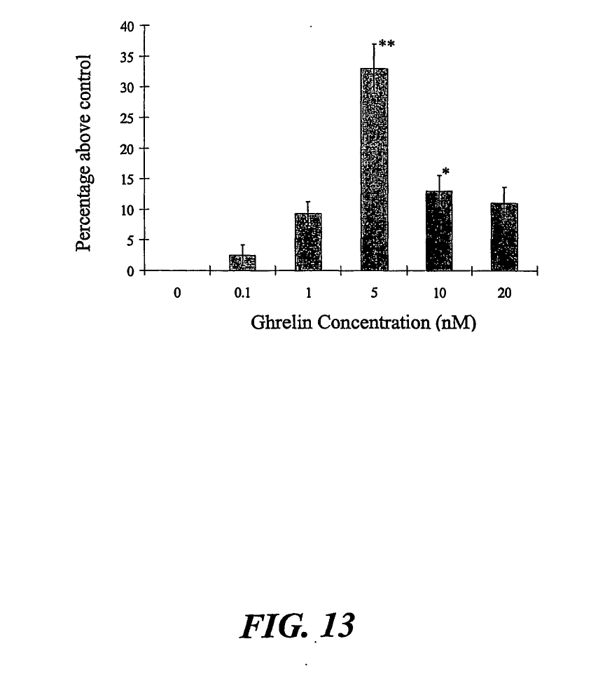

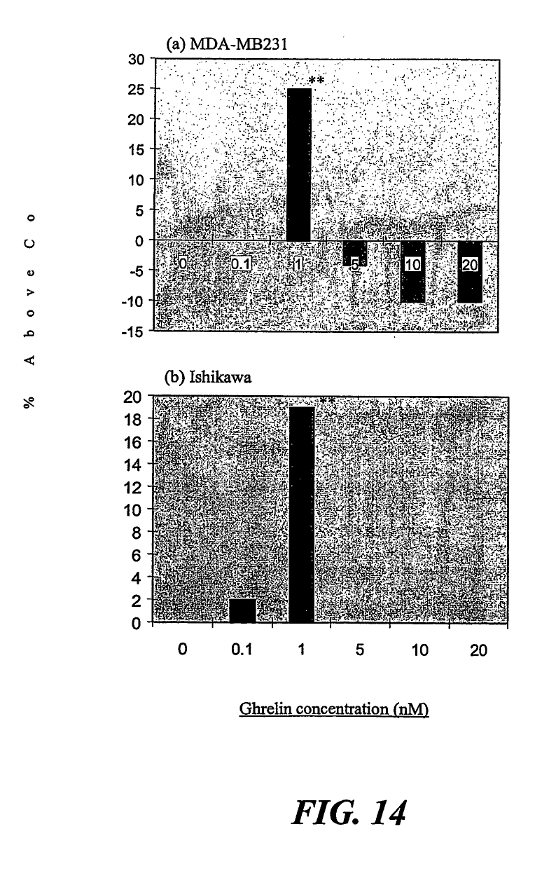

Incubation of PC3, MDA-MB 231 and Ishikawa cells with ghrelin increased cell proliferation as compared to untreated controls (FIGS. 13 and 14).

In PC3 cells, this dose-related increase peaked at 5 nM ghrelin where viable cell numbers increased to 33% above controls. In the MDA-MB 231 and Ishikawa cells, the dose related increases peaked at 25% and 19% above untreated controls respectively.

### EXAMPLE 3

### Discussion

This is the first report of the expression of ghrelin, exon 3-deleted preproghrelin and the type 1a and 1b GHS-R in human reproductive cancer cells at both the protein and nucleic acid level.

Furthermore, the present inventors have described a novel, exon 3-deleted form of preproghrelin that may have properties in its own right or the RPQPTSDRPQALLTSL (SEQ ID NO: 1) peptide may have significance after it is cleaved from the mature ghrelin (FIGS. 1 and 2).

The present invention also provides functional evidence that ghrelin may have an autocrine/paracrine role in stimulating prostate cancer cell proliferation. GHS-R expression has been demonstrated previously in the pituitary and hypothalamus (Howard et al., 1996, supra), in neuroendocrine tumours (de Keyzer et al., 1997, Eur. J. Endocrinol. 137 715) and in vitro in rat pituitary tumour cells (Adams et al., 1998, J. Clin. Endocrinol. Metab. 83 638), but not in prostate cancer. GHS-R overexpression may be associated with tumourigenesis in some tissues, as GHS-R mRNA expression is 200 fold higher in somatotroph tumours than in normal pituitary tissue (Skinner et al., 1998, J. Clin. Endocrinol. Metab. 83 4314). The type 1a and 1b GHS-R isoforms are co-expressed in central nervous system tumours (Korbonits et al., 1999, IGF Res. 9 93) and in the prostate cancer cell lines studied herein. A normal prostate cDNA library did not express GHS-R 1b transcripts and this may represent a difference between the normal and cancerous state. Although no function has yet been ascribed to the type 1b GHS-R, given the conservation of the intron-derived coding sequence between human and swine type 1b cDNAs, the isoform may have functional significance in some tissues (Van der Ploeg, 1998, In: GHSs in clinical practice (Eds Bercu & Walker pp 59-75. Springer-Verlag N.Y.).

Ghrelin mRNA expression has been demonstrated in rodent brain, some rodent peripheral tissues, human stomach (Kojima et al., 1999, supra) and human neuroendocrine tumours (Korbonits et al., 2001, J. Clin. Endocrinol. Metab. 86 881). No previous studies have examined the expression of ghrelin in peripheral cancers. In this study, ghrelin mRNA expression was demonstrated by RT-PCR in the four prostate cancer cell lines studied, but not in a normal prostate cDNA library. This could reflect a very low abundance of ghrelin mRNA in normal tissue and a relative over-expression of ghrelin in prostate cancer. Ghrelin significantly increases the proliferation of prostate cancer cells in vitro. This finding implies that ghrelin activates the (therefore functional) GHS-R 1 a receptor to induce proliferation, either by the autocrine action of secreted prostatic GH or by some other more direct signalling mechanism. The bell-shaped proliferation response curve demonstrated by the PC3 cells in response to ghrelin treatment could be a result of down-regulation of GHS-R expression by prostatic GH, as GHS-Rs in the rat hypothalamus are down-regulated by GH (Bennet et al., 1997, Endocrinology 138 4552).

The discovery that ghrelin induces a proliferative response in PC3 cells is the first demonstration of a proliferative role for ghrelin in any cell type. This finding is pertinent given that the GHRH-GH-IGF axis is a promising target for anti-tumour therapies for GH responsive cancers. Blockade of the GHS-R receptor and/or the inhibition of ghrelin activity could provide future targets for the development of cancer therapies. The use of GHS treatment in the aged, where there is a decline in GH secretion, has been proposed. In light of this study, caution is indicated regarding the use of GHSs and ghrelin in medical and non-medical settings, particularly in the aging male. Furthermore, diagnostic methods are contemplated whereby detection of ghrelin or GHS-R 1b (or encoding nucleic acids) may assist in identification of reproductive cancer cells in a patient sample.

In conclusion, this is the first study to demonstrate the co-expression of ghrelin, exon 3-deleted preproghrelin and the GHS-R in cancer cells of the reproductive system and the first to provide evidence that a previously unrecognised prostatic autocrine pathway involving ghrelin is capable of stimulating growth of prostate cancer cells in vitro.

Throughout this specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

The disclosure of each patent and scientific document, computer program and algorithm referred to in this specification is incorporated herein by reference in its entirety.

TABLE 1

| | Tissues | | | |
|---|---|---|---|---|
| | Normal | Breast | Normal | Prostate |
| Antibody | breast | cancer | prostate | cancer |
| Ghrelin | + | +++ | + | ++++ |
| Exon 3-deleted ghrelin | ++ | +++++ | + | +++++ |
| GHS-R 1b | − | ++++ | − | ++++ |

1. A method of identifying a cancer cell or tissue of the reproductive system, said method including the step of detecting expression of a ghrelin protein, an exon 3-deleted preproghrelin protein and/or a GHS-R 1b protein by a cell or tissue of the reproductive system, wherein at least the presence of said ghrelin protein, said exon3-deleted preproghrelin protein or said GHS-R 1b protein indicates that said cell or tissue is a cancer cell or tissue.

2. A method of identifying a cancer cell or tissue of the reproductive system, said method including the step of detecting expression of a ghrelin nucleic acid, an exon3-deleted preproghrelin nucleic acid and/or a GHS-R 1b nucleic acid by a cell or tissue of the reproductive system, wherein at least the presence of said ghrelin nucleic acid, said exon3-deleted preproghrelin nucleic acid or said GHS-R 1b nucleic acid indicates that said cell or tissue is a cancer cell or tissue.

3. The method of claim 1 or claim 2, wherein expression of ghrelin, exon3-deleted preproghrelin or GHS-R 1b protein or nucleic acid is higher in said cancer cell or tissue than in a corresponding normal cell or tissue.

4. The method of claim 1 or claim 2, wherein expression of GHS-R 1b protein or nucleic acid is detected as an indication that said cell or tissue is a cancer cell or tissue.

5. The method of claim 1 or claim 2, wherein expression of ghrelin protein or nucleic acid is detected as an indication that said cell or tissue is a cancer cell or tissue.

6. The method of claim 1 or claim 2, wherein expression of exon 3-deleted form of preproghrelin protein or nucleic acid is detected as an indication that said cell or tissue is a cancer cell or tissue.

7. The method of claim 1 or claim 2, wherein the cancer of the reproductive system is selected from prostate cancer, ovarian cancer, breast cancer, cervical cancer, choriocarcinoma and uterine cancer.

8. The method of claim 7, wherein the cancer of the reproductive system is prostate cancer or breast cancer.

9. The method of claim 1 or claim 2 wherein said individual is a mammal.

10. The method of claim 9 wherein said mammal is a human.

11. The method of claim 1 wherein expression of a ghrelin, exon3-deleted preproghrelin or GHS-R 1b protein is detected by immunohistochemical staining, western blotting or ELISA.

12. The method of claim 2 wherein expression of a ghrelin, exon 3-deleted preproghrelin or GHS-R 1b nucleic acid is detected by RT-PCR.

13. An isolated protein that comprises the amino acid sequence RPQPTSDRPQALLTSL (SEQ ID NO:1).

14. An exon 3-deleted form of preproghrelin that comprises the amino acid sequence:

| | |
|---|---|
| MPSPGTVCSLLLLGMLWLDLAMAGSSFLSPEHQR | (SEQ ID NO: 2) |
| VQQRKESKKPPAKLQPRALAGWLRPEDGGQAEGA | |
| EDELEVRRPQPTSDRPQALLTSL. | |

15. An antibody that is capable of binding SEQ ID NO: 1 or SEQ ID NO:2.

16. An isolated nucleic acid that encodes a protein comprising SEQ ID NO:1 or SEQ ID NO:2.

17. The isolated nucleic acid of claim 16 wherein the isolated nucleic acid has the nucleotide sequence set forth in SEQ ID NO:3.

18. The isolated nucleic acid of claim 16 which comprises nucleotides 33 to 305 of SEQ ID NO:3.

19. An expression construct comprising the isolated nucleic acid of claim 17 or claim 18 operably linked to one or more regulatory nucleotide sequences in an expression vector.

20. An expression construct comprising a ghrelin nucleic acid, an exon 3-deleted preproghrelin nucleic acid, a GHS-R 1a nucleic acid or a GHS-R 1b nucleic acid in an antisense orientation (3′→5′) operably linked to one or more regulatory nucleotide sequences in an expression vector.

21. A host cell transfected or transformed with the expression construct of claim 19 or claim 20.

22. A method of identifying an antagonist of ghrelin activity, said method including the step of determining whether a candidate antagonist inhibits or suppresses ghrelin activity.

23. A pharmaceutical composition comprising an antagonist of ghrelin activity together with a pharmaceutically-acceptable carrier, diluent or excipient.

24. A method of treating cancer of the reproductive system, said method including the step of administering to an individual an antagonist of ghrelin activity.

25. The method of claim 24, wherein the cancer of the reproductive system is selected from prostate cancer, ovarian cancer, breast cancer, cervical cancer, choriocarcinoma and uterine cancer.

26. The method of claim 26, wherein the cancer of the reproductive system is prostate cancer or breast cancer.

27. The method of claim 24 wherein cancer of the reproductive system further includes hyperproliferative disorders of the reproductive system.

28. The method of claim 27 wherein the hyperproliferative disorder is benign prostatic hyperplasia.

29. The method of claim 24 wherein said individual is a mammal.

30. The method of claim 29 wherein said mammal is a human.


SEQUENCE LISTING (17 sequences)

SEQ ID NO: 1 - protein, 16 aa, Homo sapiens
RPQPTSDRPQALLTSL

SEQ ID NO: 2 - protein, 91 aa, Homo sapiens
MPSPGTVCSLLLLGMLWLDLAMAGSSFLSPEHQRVQQRKESKKPPAKLQPRALAGWLRPEDGGQAEGAEDELEVRRPQPTSDRPQALLTSL

SEQ ID NO: 3 - DNA, 401 bp, Homo sapiens
caggcccacctgtctgcaacccagctgaggccatgccctccccagggaccgtctgcagcctcctgctcctcggcatgctctggctggacttggccatggcaggctccagcttcctgagccctgaacaccagagagtccagcagagaaaggagtcgaagaagccaccagccaagctgcagccccgagctctagcaggctggctccgcccggaagatggaggtcaagcagaaggggcagaggatgaactggaagtccggaggccccagccgacaagtgatcgcccacaagccttactcacctctctctaagtttagaagcgctcatctggcttttcgcttgcttctgcagcaactcccacgactgttgtacaagctcaggaggcgaataaatgttcaaactgt

SEQ ID NO: 4 - DNA, 510 bp, Homo sapiens
caggcccacctgtctgcaacccagctgaggccatgccctccccagggaccgtctgcagcctcctgctcctcggcatgctctggctggacttggccatggcaggctccagcttcctgagccctgaacaccagagagtccagcagagaaaggagtcgaagaagccaccagccaagctgcagccccgagctctagcaggctggctccgcccggaagatggaggtcaagcagaaggggcagaggatgaactggaagtccggttcaacgccccctttgatgttggaatcaagctgtcaggggttcagtaccagcagcacagccaggccctggggaagtttcttcaggacatcctctgggaagaggccaaagaggccccagccgacaagtgatcgcccacaagccttactcacctctctctaagtttagaagcgctcatctggcttttcgcttgcttctgcagcaactcccacgactgttgtacaagctcaggaggcgaataaatgttcaaactgt

SEQ ID NO: 5 - protein, 117 aa, Homo sapiens
MPSPGTVCSLLLLGMLWLDLAMAGSSFLSPEHQRVQQRKESKKPPAKLQPRALAGWLRPEDGGQAEGAEDELEVRFNAPFDVGIKLSGVQYQQHSQALGKFLQDILWEEAKEAPADK

SEQ ID NO: 6 - DNA, 20 bp, Homno sapiens
gtggggcgccccaggcacca

SEQ ID NO: 7 - DNA, 20 bp, Homo sapiens
ttggccttggggttcagggg

SEQ ID NO: 8 - DNA, 21 bp, Homo sapiens
tcttccttcctgtcttctgtc

SEQ ID NO: 9 - DNA, 19 bp, Homo sapiens
aagtctgaacactgccacc

SEQ ID NO: 10 - DNA, 19 bp, Homo sapiens
ccttctcccttctctctga

SEQ ID NO: 11 - DNA, 20 bp, Homo sapiens
gaggatgaactggaagtccg

SEQ ID NO: 12 - DNA, 20 bp, Homo sapiens
catttattcgcctcctgagc

SEQ ID NO: 13 - DNA, 20 bp, Homo sapiens
tgatggcagcactgaggtag

SEQ ID NO: 14 - DNA, 20 bp, Homo sapiens
ttgaaccggacttccagttc

SEQ ID NO: 15 - DNA, 18 bp, Homo sapiens
aatggagtccggaggccc

SEQ ID NO: 16 - DNA, 20 bp, Homo sapiens
gaacatttattcgcctcctg

SEQ ID NO: 17 - protein, 11 aa, Homo sapiens
RAWTESSINTC